(19) United States
(12) Patent Application Publication
(10) Pub. No.: US 2009/0012487 A1
(43) Pub. Date: Jan. 8, 2009

(54) Sanitary Napkin Containing Herb Ingredients

(75) Inventor: Sung Woo Park, Seoul (KR)

Correspondence Address: SUGHRUE MION, PLLC, 2100 PENNSYLVANIA AVENUE, N.W., SUITE 800, WASHINGTON, DC, 20037, US

(73) Assignee: CAP SCIENCE CO., LTD., Seoul (KR)

(21) Appl. No.: 12/278,608

(22) PCT Filed: May 16, 2006
(86) PCT No.: PCT/KR2006/001822

§ 371 Date: Aug. 7, 2008

(30) Foreign Application Priority Data
Feb. 8, 2006 (KR) 10-2006-0012029

(51) Int. Cl. A61F 13/15

(52) U.S. Cl. 604/359; 604/367; 604/385.23

(57) ABSTRACT

Disclosed is a sanitary napkin containing polymeric absorbents and herbal ingredients, and more particularly to a functional sanitary napkin, in which a absorbent layer structure having the polymeric absorbents and herbal ingredients distributed thereon consists of three layers of non-woven fabric (or pulp) and is surrounded by a polymeric absorbent—containing surge layer made of an air-laid material, such that the ability to absorb menstrual blood is increased and the odor of the herbal ingredients is prevented from permeating undergarments, garments, bags and the like.

### TECHNICAL FIELD

The present invention relates to a sanitary napkin containing polymeric absorbents and herbal ingredients, and more particularly to a functional sanitary napkin containing herbal ingredients, in which an absorbent layer structure having the polymeric absorbents and herbal ingredients distributed therein consists of three layers of non-woven fabric (pulp) and is surrounded by a polymeric absorbent-containing surge layer made of an air-laid material, such that the ability to absorb menstrual blood is increased and the odor of the herbal ingredients is prevented from permeating undergarments, garments, bags and the like.

### BACKGROUND ART

Sanitary napkins (or woman pads or menstrual napkins) are media for absorbing a large amount of body fluid such as menstrual blood or secretions released from the body during a women's menstrual period and are constructed such that they are worn by women. When worn by users, these are disposed on the vulva and attached to underwear by an adhesive agent or the like. Also, the sanitary napkin is designed in terms of function such that it can absorb a large amount of body fluid and can be worn for a long period of time.

This sanitary napkin generally comprises a topsheet layer, a backsheet layer and an absorbent layer interposed between the topsheet layer and the backsheet layer.

The topsheet layer is a liquid-pervious portion coming directly into contact with the vulva of the body, and the backsheet layer, which is impervious to liquid, prevents absorbed menstrual blood from leaking to the outside, and thus prevents menstrual blood from staining underwear and the like. The absorbent layer serves to absorb menstrual blood passed through the topsheet layer and is composed of an absorbent material consisting of a polymeric absorbent such as super absorbent polymer.

The polymeric absorbent is generally in the form of white powder, which, when immersed into water, instantaneously absorbs the water so as to be wetted and gelled. This polymeric absorbent generally has a water-absorbing ability of more than 100 g/g, which is about 1000-fold higher than that of general absorbent compositions, and thus it has a high water absorbing rate and can absorb a larger amount of menstrual blood, compared to general absorbent compositions.

Although the absorbent material employing this polymeric absorbent has an excellent ability to absorb and hold menstrual blood, secretions and the like, but has no antibacterial activity. Thus, there is a problem in that, when the polymeric absorbent absorbs menstrual blood, secretions and the like, organic substances contained in the absorbed liquid will be degraded by bacteria or microorganisms, so that the products resulting from the degradation will cause an offensive odor, bacterial proliferation, skin irritation or the like.

In an attempt to solve said problem, Korean Patent Application Nos. 10-2003-0047258 and 10-2003-0089804 disclose a sanitary napkin (woman pad) having offensive odor-preventing and antibacterial functions, which contains a functional herbal composition as an absorbent composition.

However, the sanitary napkin containing said functional composition has problems in that, because a unique odor caused by herbal powder permeates undergarments, garments, bags and the like, the sanitary napkin can cause an unpleasant sensation to a user and it is unsuitable to carry the sanitary napkin at ordinary times.

### DISCLOSURE OF INVENTION

### Technical Problem

The present invention has been made to solve the above-described problems occurring in the prior art, and it is an object of the present invention to provide a sanitary napkin in which an absorbent layer structure having a polymeric absorbent and herbal powder distributed thereon is made of three layers of non-woven fabric (or pulp) so as to increase the absorbent ability of the sanitary napkin and to prevent herbal odor from permeating undergarments.

Another object of the present invention is to provide a functional sanitary napkin, which comprises a surge layer made of an air-laid material, which surrounds said absorbent layer structure, so as to further increase the absorbent ability and the function of preventing odor permeation.

Still another object of the present invention is to provide a functional sanitary napkin, in which a polymeric absorbent sheet containing herbal powder is located at a central absorbent region, such that the sanitary napkin is thicker at the central portion so as to more rapidly absorb menstrual blood and to prevent absorbed menstrual blood from leaking to both sides.

### Technical Solution

To achieve the above objects, the present invention provides a sanitary napkin containing herbal ingredients, which comprises a liquid-pervious topsheet layer, a liquid-impervious backsheet layer, and an absorbent layer structure interposed between the topsheet layer and the backsheet layer so as to absorb menstrual blood and secretions, wherein said absorbent layer structure is made of three layers of non-woven fabric (or pulp), and a polymeric absorbent and herbal powder are uniformly distributed between the non-woven fabric layers (or pulp layers).

In the inventive sanitary napkin, a surge layer having a polymeric absorbent distributed therein is preferably disposed so as to surround the absorbent layer structure.

Also, the polymeric absorbent and the herbal powder are preferably mixed with each other at a weight ratio of 55-60 (polymeric absorbent): 45-40 wt % (herbal powder).

Also, 28-32 g of the polymeric absorbent and 19-23 g of the herbal powder are uniformly distributed per 1 m³ of the absorbent layer structure.

Also, the herbal powder is preferably a mixture of 11-13 wt % of Leonurus sibiricus, 6-8 wt % of Cyperus rotundus, 6-8 wt % of Saururus chinensis, 28-30 wt % of mugwort, 18-20 wt % of Cnidium officinale Makino, 11-13 wt % of peppermint, and 13-15 wt % of Angelica gigas.

In addition, said absorbent layer structure is preferably disposed at the central portion of the sanitary napkin along the longitudinal central axis such that the central portion is thicker than both sides.

### ADVANTAGEOUS EFFECTS

According to the above-described construction of the present invention, the absorbent layer structure in the inventive sanitary napkin consists of three layers and is surrounded by the polymeric absorbent-containing surge layer. Thus, the inventive sanitary napkin can more rapidly and strongly absorb body secretions.

Also, the inventive sanitary napkin has an increased antibacterial activity, because the herbal ingredients inhibit the proliferation of bacteria and kills proliferated bacteria.

Also, in the functional sanitary napkin according to the present invention, the herbal powder is applied between the three non-woven fabric layers (or pulp layers) constituting the absorbent layer structure, and the absorbent layer structure is surrounded by air-laid. Thus, the herbal odor is prevented from permeating undergarments, bags and the like.

Furthermore, the functional sanitary napkin according to the present invention is thicker at the central portion than both sides thereof due to the thickness of the absorbent layer structure consisting of three layers. Thus, when the inventive sanitary napkin is worn by a user, it will adhere more closely to the vulva of the body, while it will more rapidly absorb menstrual blood, and the absorbed menstrual blood will not leak to the outside. Thus, the topsheet layer will be always maintained in a dry state, so that the sanitary napkin will have a good wearing sensation, while it will not cause skin troubles.

In addition, the functional sanitary napkin according to the present invention has excellent effects of relieving menstrual cramps due to the pharmacological action of the herbal ingredients, and excellent effects of neutralizing odor due to the herbal fragrance.

### BRIEF DESCRIPTION OF THE DRAWINGS

FIG. 1 is a perspective view showing the disassembled state of a functional sanitary napkin according to the present invention.

FIG. 2 is a perspective view showing the functional sanitary napkin shown in FIG. 1.

FIG. 3 is a cross-sectional view taken along line A-A′ of FIG. 1.

FIG. 4 is a top view of the functional sanitary napkin shown in FIG. 1.

### LIST OF REFERENCE NUMERALS

10: polymeric absorbent;
20: herbal powder;
100: sanitary napkin;
110: flap portion;
120: flap adhesive layer;
130: flap release paper;
140: intermediate adhesive layer;
150: release paper;
160: lower adhesive layer;
170: packaging sheet;
180: adhesive tape;
210: topsheet layer;
220: impervious layer;
230: air-permeable layer;
240: absorbent layer structure;
241: first non-woven fabric (or pulp) layer;
242: second non-woven fabric (or pulp) layer;
243: third non-woven fabric (or pulp) layer; and
250: surge layer.

### BEST MODE FOR CARRYING OUT THE INVENTION

Hereinafter, a preferred embodiment of the present invention will be described in detail with reference to the accompanying drawings.

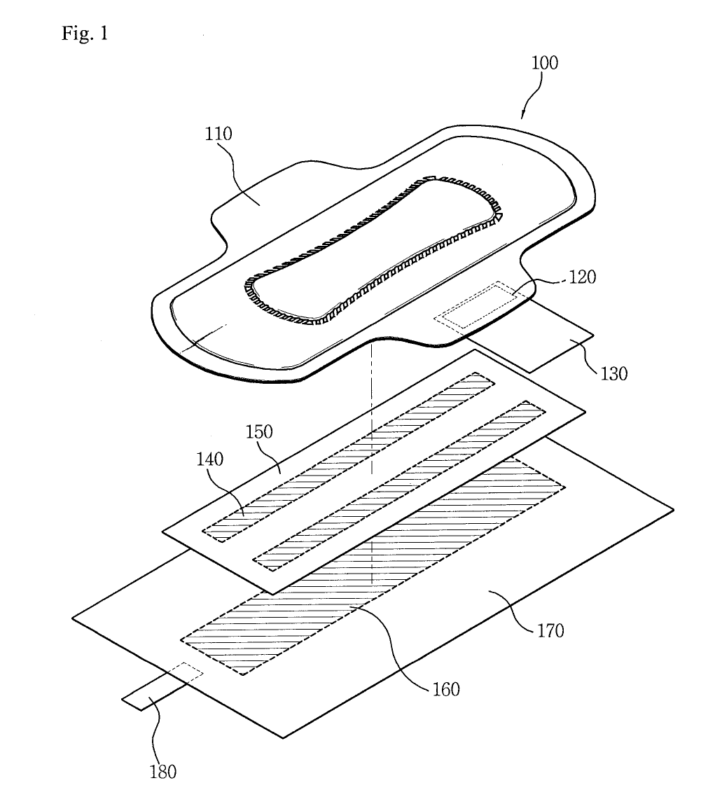

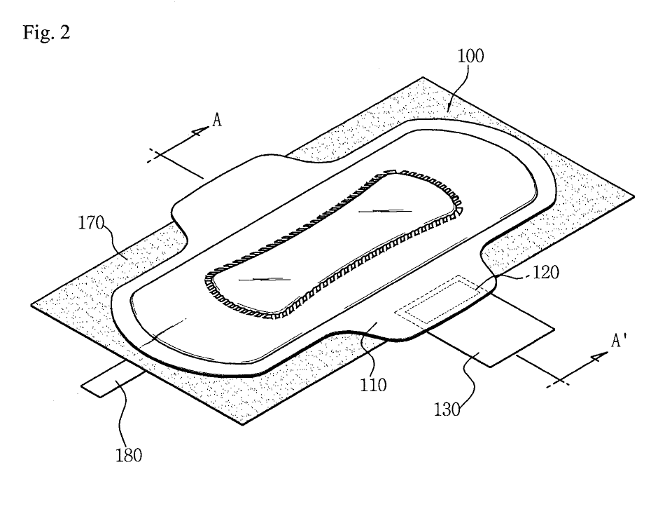

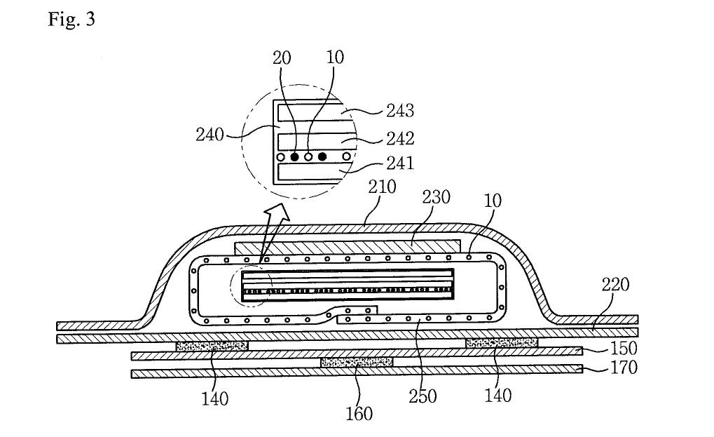

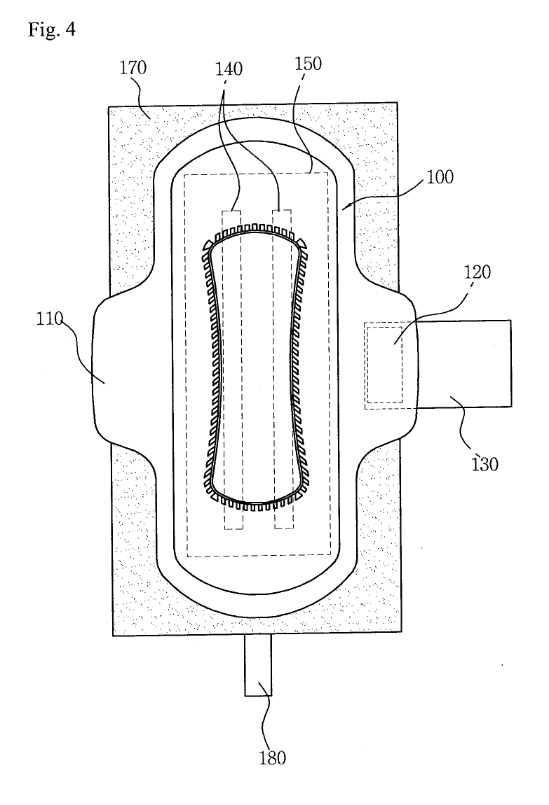

FIG. 1 is a perspective view showing the disassembled state of a functional sanitary napkin according to the present invention, FIG. 2 is a perspective view showing the functional sanitary napkin shown in FIG. 1, FIG. 3 is a cross-sectional view taken along line A-A′ of FIG. 1, and FIG. 4 is a top view of the functional sanitary napkin shown in FIG. 1.

As shown in the figures, a sanitary napkin 100 according to an embodiment of the present invention has the shape of a known sanitary napkin and is designed such that it absorbs body fluids such as menstrual blood and other secretions, which are released during a women's menstrual period. It is classified, according to its intended use, into large-size, middle-size, small-size and panty liner products, and is also classified, according to its shape, into a general type and a flap type having flaps 120 formed at both sides thereof.

To the lower outer surface of the sanitary napkin 100, a release paper 150 is bonded by means of an intermediate adhesive layer 140, and to the lower outer surface of the release paper 150, a packaging sheet 170 is bonded by means of a lower adhesive layer 160, so that the sanitary napkin 100 and the release paper 150 are easily separated from each other. Also, in the case of the flap-type sanitary napkin, a flap adhesive layer 120 is formed on the outer surface of the flaps 110 provided at both sides of the sanitary napkin, and a flap release paper 130 is bonded to the flaps such that it can be easily separated from the flaps.

The sanitary napkin 100 having the above-described structure is individually packaged by the packaging sheet 170 such that it is easy to carry. On one side of the packaging sheet 170, an adhesive tape 180 is preferably provided, such that the packaging sheet 170 is easily separated out upon the use of the sanitary napkin 100. Moreover, on the upper portion of the sanitary napkin 100, an embossing line for increasing absorbent ability and preventing leakage can also be formed.

As shown in FIG. 3, the sanitary napkin 100 comprises a liquid-pervious topsheet layer 210, a liquid-impervious backsheet layer 220, an air-permeable layer 230 laminated on the lower surface of the topsheet layer 210, an absorbent layer structure interposed between the topsheet layer 210 and backsheet layer 220, and a surge layer 250 surrounding around the absorbent layer structure 240.

The topsheet layer 210 is a liquid-pervious uppermost layer coming directly into contact with the vulva of the body and has a plurality of pores formed therein such that it can directly absorb body secretions. The topsheet layer 210 is generally made of, for example, a non-woven fabric or polyethylene mesh film.

The backsheet layer 220 functions to prevent absorbed secretions from leaking to the outside, and has a liquid-impervious surface formed on the lower surface thereof. The backsheet layer 220 is made of an air-permeable polyethylene (PE) film or a non-woven fabric laminated on the polyethylene film.

The air-permeable layer 230 serves to rapidly and uniformly transfer absorbed menstrual blood into the absorbent layer structure 240 and is made of a bulky non-woven fabric or tissue.

The absorbent layer structure 240 is made of a liquid-pervious non-woven fabric or pulp material capable of absorbing secretions such as blood and contains super absorbent polymer (SAP) and the like. In the embodiment of the present invention, a polymeric absorbent 10 and an herbal powder 20 are coated on the absorbent layer structure 240.

As shown in a circular enlarged view in FIG. 3, the absorbent layer structure 240 is composed of three non-woven fabric (pulp) layers consisting of a first non-woven fabric layer (or pulp layer) 241, a second non-woven fabric layer (or pulp layer 242 and a third non-woven fabric layer (or pulp layer) 243, starting with the lowest layer. Between the first non-woven fabric layer (or pulp layer) 241 and the second non-woven fabric layer (or pulp layer) 242, the polymeric absorbent 10 and the herbal powder 20 are uniformly distributed.

Because the absorbent layer structure 240 consists of three layers as described above, the sanitary napkin 100 has an increased absorbent ability, and because the herbal powder 20 is applied between the three non-woven fabric layers (or pulp layers) constituting the absorbent layer structure 240, the unique odor of the herbal powder is prevented from permeating hand, undergarments, garments, bags and the like during the carrying or use of the sanitary napkin 100. Also, due to the thickness of the absorbent layer structure 240 consisting of three layers, the sanitary napkin is thicker at the central portion thereof than both sides, so that the sanitary napkin more rapidly absorbs menstrual blood and prevents the absorbed menstrual blood from leaking, while it adheres more closely to the vulva of the body when worn by a user.

In the embodiment of the present invention, the polymeric absorbent 10 and the herbal powder 20 are applied between the first non-woven fabric layer (or pulp layer) and 241 and the second non-woven fabric layer (or pulp layer) 242 among the three non-woven (or pulp) layers. However, if necessary, the polymeric absorbent 10 and the herbal powder 20 may also be applied between the second non-woven fabric layer (or pulp layer) 242 and the third non-woven fabric layer (or pulp layer) 243 or between all the non-woven fabric layers (or pulp layers). Moreover, in addition to the three-layer structure, the absorbent layer structure 240 may also consist of two layers between which the polymeric absorbent 10 and the herbal powder 20 are applied. Also, the absorbent layer structure may consist of one layer or four or more layers.

The polymeric absorbent 10 and the herbal powder 20, which are applied as described above, are preferably mixed with each other at a weight ratio of 55-60 (polymeric absorbent 10): 45-40 (herbal powder 20). Also, based on 1 m³ of the absorbent layer structure 240, 28-32 g of the polymeric absorbent and 19-23 g of the herbal powder are uniformly distributed on the absorbent layer structure.

The polymeric absorbent 10 used in the sanitary napkin 100 can generally absorb blood about 1,000 times its own weight. Also, the polymeric absorbent 10 has not only rapid absorption ability, but also a strong liquid-binding ability. Thus, after menstrual blood absorbed into the polymeric absorbent is changed into gel, it does not flow outward even under the application of pressure.

The herbal powder 20 consists of herbal ingredients such as Leonurus sibiricus, Cyperus rotundus, Saururus chinensis, mugwort, Cnidium officinale Makino, peppermint and Angelica gigas. The effects and pharmacological actions of the herbal ingredients are as follows.

Leonurus sibiricus

The leaf of Leonurus sibiricus contains components such as leonurine, leonuridine rutin, vitamin A and fatty oil, and it has an excellent effect of regulating menstruation, and thus has been used for the treatment of women's diseases such as irregular menstruation and hypochondriac pain due to blood stagnation after childbirth.

Cyperus rotundus

The dried tuber of Cyperus rotundus is used for pharmacological purposes, is rich in cyperene, cyperol, isocyperene, sugenol, kobusone and fatty acid, and acts to relax pain or myometrium contraction. In Chinese medicine, it is formulated for the treatment of irregular menstruation, menstrual cramps, hysteria, menopausal troubles and the like.

Saururus chinensis

Saururus chinensis contains water soluble tannin and flavone-based substances, including quercitrin, isoquercitrin and flavonoid. Due to the action of these components, it cleans blood, strengthens capillary vessels, makes blood circulation smooth, and inhibits the coagulation of platelets. Thus, it acts against various adult diseases such as arterosclerosis, has effects against various women's diseases, and particularly has effects on the treatment of leucorrhea, uteritis, irregular menstruation, uterovaginal prolapse and the like.

Mugwort

Mugwort mainly contains 50% cineol, adenine, choline, tannin, chlorophyll and the like, acts to alleviate a fever and to promote blood circulation, and has an effect of warming the body. Also, it acts to stop uterine hemorrhage, hemetemesis and hemoptysis, is frequently used as a drug against abdominal coldness and pain, menstruation irregularity and menstrual cramps, and has a deodorizing effect.

Cnidium officinale Makino

The stem and root of Cnidium officinale Makino are used as medical drugs and contain 1-2% essential oil and amino acid. In Chinese medicine, it is frequently used as blood-replenishing, blood-activating and blood-cleaning agents for the treatment of women's diseases. It has effects on the treatment of menstruation irregularity, menstrual cramps, amenorrhea, and various diseases before or after childbirth, increases uterine contraction and dilatation abilities. Also, it acts to relieve convulsions, and thus shows excellent effects as a pain-killer and a sedative.

Peppermint

Peppermint mainly contains menthol, menthone, camphen, limonene and the like, alleviates pain, provides cool sensations, alleviates clods and fever, and has effects on anti-inflammation, capillary vessel dilatation, uterine contraction increase, pain alleviation and the like.

Angelica gigas

Angelica gigas contains decursin and decurcinol as main components and is widely formulated as a typical blood-replenishing drug in various women's diseases. It has effects on sedation, blood replenishment, anti-vitamin E activity, myometrium contraction and relaxation, and thus is used for the treatment of menstruation irregularity and menstrual cramps. Also, it is characterized in that it has a unique and strong fragrance.

The herbal ingredients as described above are uniformly coated on the absorbent layer structure 240 in the form of the herbal powder consisting of a mixture of 11-13 wt % of Leonurus sibiricus, 6-8 wt % of Cyperus rotundus, 6-8 wt % of Saururus chinensis, 28-30 wt % of mugwort, 18-20 wt % of Cnidium officinale Makino, 11-13 wt % of peppermint, and 13-15 wt % of Angelica gigas.

Also, the central absorbent region constituting the absorbent layer structure 240 is disposed at the central portion of the sanitary napkin 100 along the longitudinal central axis. For this reason, the sanitary napkin 100 is slightly thicker at the central portion than both sides thereof. Accordingly, when the sanitary napkin 100 is worn by a user, the central portion that absorbs menstrual blood will adhere closely to the vulva of the body, and thus the menstrual blood will be more rapidly absorbed into the sanitary napkin.

The surge layer 250 surrounds around the absorbent layer structure 240 having the polymeric absorbent 10 and herbal powder 20 coated thereon. It consists of an air-laid material and has a polymeric absorbent 10 uniformly coated therein.

The air-laid material is prepared from pulp as a main material using compressed air together with an adhesive agent, and has a high volume per unit weight and an excellent absorbent ability, because there is no great difference in the transversal and longitudinal tensile strengths thereof. Thus, it is widely used in hygienic products (diapers and sanitary napkins) requiring absorbent ability.

As described above, the absorbent layer structure 240 consisting of three layers is surrounded by the surge layer 250 containing the polymeric absorbent 10 so as to form a five-layer absorbent structure. Thus, menstrual blood unabsorbed into the absorbent layer structure 240, if any, can be absorbed into the surge layer 250. Accordingly, the sanitary napkin 100 can absorb more menstrual blood and can prevent the absorbed menstrual blood from leaking laterally.

Also, because the absorbent layer structure 240 having the herbal powder 20 distributed thereon is surrounded by the surge layer 250 made of air-laid material, the herbal odor emitted from the herbal composition is further blocked by the air-laid material, such that the unique odors of the herbal plants are prevented from permeating undergarments, garments, bags, hand and the like.

The function and operation of the present invention having the above construction will now be described.

When the functional sanitary napkin 100 according to present invention is worn by a user, menstrual blood and virginal secretions released from the body are absorbed into the topsheet layer 210 made of an absorbent material having a plurality of pores therein, and the absorbed secretions are passed through the air permeable layer 230 and absorbed into the absorbent layer structure 240 having the polymeric absorbent 10 and herbal powder 20 distributed thereon. Secretions unabsorbed into the absorbent layer structure 240 are absorbed into the surge layer 250, such that the menstrual blood and the like are prevented from leaking laterally. Also, the backsheet layer 220, the lowest layer, prevents the menstrual blood from staining garments and the like. In this case, the sanitary napkin 100 shows an increased absorbent ability, because the absorbent layer structure 240 consists of three non-woven fabric (or pulp) layers, the polymeric absorbent 10 is coated between the three layers, and the polymeric absorbent 10 is also coated onto the surge layer 250 surrounding around the absorbent layer structure 240.

On Nov. 28, 2005, the applicant tested absorption ratio for the product according to the present invention and commercially available products manufactured by other companies. The test results are shown in Table 1 below. As used herein, the term “absorption ratio” refers to an increase (times) in sample weight, as measured by cutting a given amount of a sanitary napkin, placing the cut napkin in water, taking out and weighing the immersed napkin after a given time period.

TABLE 1

| | | Inventive | A company's | B company's | C company's | D company's |
|---|---|---|---|---|---|---|
| | Test items | product | product | product | product | product |
| 1 | Absorption ratio | 26.7 | 16.7 | 7.7 | 12.3 | 13.3 |
| | (times) | | | | | |
| 2 | Index | 100 | 63.3 | 28.8 | 46.1 | 49.8 |

As can be seen in Table 1, the sanitary napkin 100 according to the present invention shows an absorption ratio of 26.7 times, which is significantly higher than those of the other company's products, suggesting that the sanitary napkin 100 has an excellent absorbent ability.

The absorbed organic material such as menstrual blood generally increases the proliferation of bacteria associated with biological action and causes an unpleasant odor due to degradation products and the like generated during this process. This unpleasant odor and proliferated bacteria are removed by the potent antibacterial activity of the herbal composition.

To prove this antibacterial activity, the functional sanitary napkin 100 according to the present invention was tested for deodorizing and antibacterial effects in FITI Testing Research Institute on Dec. 7, 2005. The test procedure and results are as followed.

First, the test of deodorization rate was performed according to a gas detector tube method using ammonia gas in the following manner. 100 cm² of the sample of the sanitary napkin 100 according to the present invention was placed in a gas back containing 3 liters of ammonia gas, and the concentration of ammonia gas remaining in the test bag after 2 hours was measured. As a result, more than 99.8% of the odor was removed.

Deodorization rate (%)=((Cb−Cs)/Cb)×100

wherein Cb represents the concentration of the test gas remaining in the test bag after 2 hours in the case of a blank; and Cs (sample) represents the concentration of the test gas remaining in the test bag after 2 hours in the case of the sample.

Also, the test of antibacterial activity was performed in the following manner. A given amount of Staphylococcus aureus was dropped onto a blank and the sanitary napkin 100 (sample) according to the present invention and cultured for 18 hours. Then, the number of the remaining bacteria was measured to determine the reduction rate of the bacteria, and the measurement results are shown in Table 2 below.

TABLE 2

| | Blank | Sample |
|---|---|---|
| Initial number of bacteria | 2.4 × 10⁴ | 2.4 × 10⁴ |
| After 18 hours | 4.8 × 10⁶ | 3.1 × 10³ |
| Reduction rate (%) of bacteria | — | 99.9 |

As can be seen in Table 2 above, the functional sanitary napkin 100 according to the present invention shows a bacterial reduction rate of 99.9%, suggesting that all bacteria are killed after a given time.

Thus, the unique odor of the herbal composition will not permeate undergarments or bags, because the absorbent layer structure 240, on which the herbal powder 20 having deodorizing and antibacterial effects as described above is distributed, consists of three non-woven fabric (or pulp) layers and is surrounded by the surge layer 250.

Also, due to the thickness of the absorbent layer structure 240 consisting of three layers, the sanitary napkin is thicker at the central portion than both sides thereof. Thus, when the inventive sanitary napkin is worn by a user, it will adhere more closely to the vulva of the body, while it will more rapidly absorb menstrual blood, and the absorbed menstrual blood will not leak from the napkin. Thus, the topsheet layer 210 will be always maintained in a dry state.

As described above, the functional sanitary napkin 100 containing herbal ingredients acts to alleviate menstrual cramps and prevent skin troubles, due to the effects and pharmacological actions of various herbal ingredients contained in the herbal powder 20.

During a period from Nov. 29, 2005 to Dec. 10, 2005, for the purpose of product development, the applicant conducted a survey on the satisfaction of the prevention of skin troubles and the alleviation of menstrual cramps, resulting from the use of the functional sanitary napkin 100 according to the present invention. The survey results are shown in Table 3 below. In this survey, the satisfaction for the prevention of skin troubles and the alleviation of menstrual cramps, resulting from the use of the inventive sanitary napkin, was evaluated by nine women (A-I) suffering from menstrual cramps, and was assessed as a score ranging from 0 to 5.

TABLE 3

| | | | | | | | | | | | | Score of more |
|---|---|---|---|---|---|---|---|---|---|---|---|---|
| No. | Survey items | A | B | C | D | E | F | G | H | I | Average score | than 4 (%) |
| 1 | Prevention of skin | 5 | 4 | 4 | 4 | 4 | 4 | 4 | 4 | 4 | 4.11 | 100 |
| | troubles | | | | | | | | | | | |
| 2 | Alleviation of | 5 | 5 | 4 | 3 | 3 | 4 | 5 | 3 | 4 | 4.00 | 66.7 |
| | menstrual cramps | | | | | | | | | | | |

As can be seen in Table 3 above, the inventive sanitary napkin showed an average score of 4.11 with respect to the prevention of skin troubles, and the ratio of the users who imparted a score of more than 4 (satisfaction) was 100%, suggesting that the inventive sanitary napkin 100 satisfied all the users with respect to the prevention of skin troubles. Thus, it can be seen that the functional sanitary napkin 100 according to the present invention adheres closely to the body in the use thereof so as to show good wearing sensations while acting to prevent skin troubles, because the absorbent layer structure 240 consists of three layers so as to be slightly thick at the central portion thereof.

Also, regarding the alleviation of menstrual cramps, 67% of the users answered that the inventive sanitary napkin had an effect on the alleviation of menstrual cramps. Thus, the inventive sanitary napkin is expected to alleviate menstrual cramps, because it contains herbal ingredients.

Although a preferred embodiment of the present invention has been described for illustrative purposes, those skilled in the art will appreciate that various modifications, additions and substitutions are possible, without departing from the scope and spirit of the invention as disclosed in the accompanying claims.

1. A functional sanitary napkin containing herbal ingredients, which comprises a liquid-pervious topsheet layer, a liquid-impervious backsheet layer, and an absorbent layer structure interposed between the topsheet layer and the backsheet layer so as to absorb menstrual blood and secretions, wherein said absorbent layer structure is made of three layers of non-woven fabric (or pulp), and a polymeric absorbent and herbal powder are uniformly distributed between the non-woven fabric layers (or pulp layers).

2. The functional sanitary napkin of claim 1, wherein a surge layer having a polymeric absorbent distributed therein is provided so as to surround so as to surround the absorbent layer structure having the polymeric absorbent and herbal powder distributed thereon.

3. The functional sanitary napkin of claim 1, wherein the polymeric absorbent and the herbal powder are mixed with each other at a weight ratio of 55-60 (polymeric absorbent): 45-40 (herbal powder).

4. The functional sanitary napkin of claim 1, wherein 28-32 g of the polymeric absorbent and 19-23 g of the herbal powder are uniformly distributed per 1 m³ of the absorbent layer.

5. The functional sanitary napkin of claim 1, wherein the herbal powder is a mixture of 11-13 wt % of Leonurus sibiricus, 6-8 wt % of Cyperus rotundus, 6-8 wt % of Saururus chinensis, 28-30 wt % of mugwort, 18-20 wt % of Cnidium officinale Makino, 11-13 wt % of peppermint, and 13-15 wt % of Angelica gigas.

6. The functional sanitary napkin of claim 1, wherein said absorbent layer structure is disposed at the central portion of the sanitary napkin along the longitudinal central axis such that the central portion is thicker than both sides.

7. The functional sanitary napkin of claim 2, wherein the herbal powder is a mixture of 11-13 wt % of Leonurus sibiricus, 6-8 wt % of Cyperus rotundus, 6-8 wt % of Saururus chinensis, 28-30 wt % of mugwort, 18-20 wt % of Cnidium officinale Makino, 11-13 wt % of peppermint, and 13-15 wt % of Angelica gigas.

8. The functional sanitary napkin of claim 3, wherein the herbal powder is a mixture of 11-13 wt % of Leonurus sibiricus, 6-8 wt % of Cyperus rotundus, 6-8 wt % of Saururus chinensis, 28-30 wt % of mugwort, 18-20 wt % of Cnidium officinale Makino, 11-13 wt % of peppermint, and 13-15 wt % of Angelica gigas.

9. The functional sanitary napkin of claim 4, wherein the herbal powder is a mixture of 11-13 wt % of Leonurus sibiricus, 6-8 wt % of Cyperus rotundus, 6-8 wt % of Saururus chinensis, 28-30 wt % of mugwort, 18-20 wt % of Cnidium officinale Makino, 11-13 wt % of peppermint, and 13-15 wt % of Angelica gigas.
